(19) United States
(12) Patent Application Publication
(10) Pub. No.: US 2026/0234552 A1
(43) Pub. Date: Aug. 13, 2026

(54) AVIAN STEM CELLS FOR THE PRODUCTION OF A FOOD PRODUCT

(71) Applicant: VITAL MEAT, Sèvremoine (FR)

(72) Inventor: Etienne DUTHOIT, Clisson (FR)

(21) Appl. No.: 19/639,978

(22) Filed: Apr. 6, 2026

Related U.S. Application Data
(62) Division of application No. 17/641,494, filed on Sep. 10, 2020.

(30) Foreign Application Priority Data
Sep. 10, 2019 (EP) 19196590.4

(51) Int. Cl. C12N 5/0735; A23J 3/04; A23J 3/22; A23L 13/50

(52) U.S. Cl. CPC C12N 5/0606; A23J 3/04; A23J 3/227; A23L 13/50

(57) ABSTRACT

The present invention relates to a food product comprising avian stem cells and at least one food component.

### CROSS REFERENCE TO RELATED APPLICATIONS

The present application is a divisional of U.S. application Ser. No. 17/641,494, filed on Mar. 9, 2022, which is a national stage entry of International Patent Application No. PCT/EP2020/075421, filed on Sep. 10, 2020, which claims priority to European Patent Application 19196590.4, filed on Sep. 10, 2019.

### OBJECT OF THE INVENTION

The present invention relates to a food product comprising avian stem cells and at least one food component.

### TECHNICAL BACKGROUND

In recent years, the food industry has been facing a number of challenges encouraging it to rethink methods of production for the future. The main challenges are to respond to a growing food demand due to the increase of the population while reducing the negative consequences of production on the environment. Among the concerned sectors, the meat industry is one of the most impacted as the global demand for meat should double by 2050. The supply of the world population with proteins is thus one of the major issues for the future.

Conventional farming methods use 60% of farmland and animal feed crops account for 15% to 20% of greenhouse gases. Moreover, 60 billion animals are killed per year. By 2050, the number of animals killed per year is expected to rise to 110 billion.

In addition, the development of antibiotic resistance, which reduces the possibilities of treatment in case of infection, has become a major concern in terms of human and animal health. This also contributes to the loss of consumer confidence in these traditional farming methods.

Alternative solutions to farm animals already exist such as algae consumption, insects in snacking or as animal feed, or more conventionally, the use of plant-based meat.

Plant-based meat are substitutes for meat from restructured vegetable proteins and generate a growing interest in the consumer. By way of example, WO2015/161099 describes such plant-based meat structured protein products. Plant-based meat sales in the United States of America increased by 6% in 2017 and by 24% in 2018 while the sale growth of animal meat in the United States of America grew by 2% in 2018.

However, plant-based meats do not have nutritional profiles similar to those of their animal-based counterparts. In particular, relying only on vegetable proteins as a source of proteins may lead to a deficiency in certain essential amino-acids, in particular lysine. Plant-based meats may also lack key minerals, such as iron and zinc, as well as certain vitamins, such as vitamin B12.

It is therefore necessary to develop alternative meat substitutes to meet current food issues i.e. a healthy and safe food product, having a nutritional profile level similar to meat products derived from animals, while having a reduced environmental footprint and avoiding the slaughter of animals.

### SUMMARY OF THE INVENTION

The present invention arises from the unexpected finding by the inventors that a food product obtained by at least one step of in vitro culture of avian stem cells provides high quality proteins.

Accordingly, the present invention relates to a food product, in particular a manufactured food product, comprising avian stem cells and at least one food component.

The present invention also relates to the use of avian stem cells and at least one food component for the manufacture of a food product.

The present invention also relates to a method for the preparation or the manufacture of a food product, comprising a step of mixing avian stem cells with at least one food component.

The present invention also relates to a food product liable to be obtained, or obtained, by the method as described above.

### DETAILED DESCRIPTION OF THE INVENTION

On a preliminary basis, we recall that as intended herein, the term “comprising” has the meaning of “including” or “containing”, which means that when an object “comprises” one or several elements, other elements than those mentioned may also be included in the object. In contrast, when an object is said to “consist of” one or several elements, the object is limited to the listed elements and cannot include other elements than those mentioned.

Besides, as is intended herein the expression “cultured meat” will be considered equivalent to the expressions “synthetic meat”, “clean meat”, “in vitro meat” or “cell-based meat”.

### Obtention of Avian Stem Cells

Preferably, the avian stem cells in the food product according to the invention are avian embryonic stem cells isolated from an embryo. Preferably also, the avian stem cells according to the invention are continuous diploid avian cell lines. Preferably, the avian stem cells, in particular the avian embryonic stem cells, according to the invention consist essentially of undifferentiated cells. Preferably, the avian stem cells, in particular the avian embryonic stem cells, according to the invention consist of at least 50% of undifferentiated cells. More preferably, the avian stem cells, in particular the avian embryonic stem cells, according to the invention consist of at least 60%, at least 70%, at least 80%, at least 90, at least 95% of undifferentiated cells.

Preferably, the avian stem cells, in particular the avian embryonic stem cells, according to the invention are obtained by a method comprising at least one step of in vitro culture. Preferably, the avian stem cells, in particular the avian embryonic stem cells, according to the invention are obtained by a method comprising the following steps:
a) Isolation, culture and expansion of avian stem cells, in particular avian embryonic stem cells, in a complete culture medium containing. Preferably the complete culture medium according to the invention comprises factors allowing growth the avian stem cells, in particular the embryonic avian stem cells. Preferably, the culture and expansion of avian stem cells, in particular avian embryonic stem cells, according to the invention is done in the presence of a feeder layer and supplemented with animal serum. Optionally, said complete culture medium may comprise additives, such as amino-acids (i.e. glutamine, non-essential amino acids, etc.), sodium pyruvate, beta-mercaptoethanol, vitamins, protein hydrolysate of non-animal origin (i.e. yeastolate, plant hydrolysates soy, wheat, etc.).
b) Passage by modifying the culture medium so as to obtain a total withdrawal of said factors, said feeder layer and said serum, and optionally said additives, and further obtaining adherent or suspension avian stem cells, in particular avian embryonic stem cells, capable of proliferating over a long period of time, in a basal medium in the absence of exogenous growth factors, feeder layer and animal serum.

The term “avian” as used herein is intended to refer to any species, subspecies or race of organism of the taxonomic class “ava”, such as, but not limited to, chicken, turkey, duck, goose, quails, pheasants, parrots, finches, hawks, crows, ostrich, emu and cassowary. The term “avian”, “bird”, “aves” or “ava” as used herein is intended to have the same meaning, and will be used indistinctly. In a preferred embodiment, “birds” refer to any animal of the taxonomic order:
“Anseriformes” (i.e duck, goose, swan and allies). The order Anseriformes contains about 150 species of birds in three families: the Anhimidae (the screamers), Anseranatidae (the Magpie-goose), and the Anatidae, which includes over 140 species of waterfowl, among them the ducks, geese, and swans. All species in the order are highly adapted for an aquatic existence at the water surface. All are web-footed for efficient swimming (although some have subsequently become mainly terrestrial). The term includes the various strains of ducks, for example Pekin duck and Muscovy duck.
“Galliformes” (i.e chicken, quails, turkey, pheasant and allies). The Galliformes is an order of birds containing the chicken, turkeys, quails and pheasants. About 256 species are found worldwide. The term includes the various strains of Gallus gallus, or chickens, for example S86N, Valo, White Leghorn, Brown Leghorn, Sussex, New Hampshire, Rhode Island, Ausstralorp, Minorca, Amrox, California Gray, East Lansing, Italian-Partridge-colored, Marans, Barred Rock, Cou Nu Rouge (CNR), GF30, ISA as well as strains of turkeys, pheasants, quails, and other poultry commonly bred.
“Columbiformes” (i.e Pigeon and allies). The bird order Columbiformes includes the very widespread doves and pigeons.

Preferably, avian embryonic stem cells according to the invention are stem cells which have the characteristic feature of being obtained from culturing parts or all of a very early embryo (e.g blastula stage). These embryonic stem cells preferably exhibit in vitro all the characteristics of a stem cell, and in vivo the unique capacity of contributing to the morphogenesis of an embryo and of participating in germline colonization when they are re-implanted in any manner whatsoever in a recipient embryo. Primordial Germ Cells (PGC) which are the progenitors of the sperm or ovocyte cells developed after sexual maturity are pluripotent embryonic stem cells and constitutes a subtype of embryonic stem cells.

In a preferred embodiment, the avian embryonic stem cells of the present invention are chicken embryonic stem cells. The chicken embryonic stem cells according to the invention are preferably selected from the group consisting of chicken strains DF-1, S86N, Valo, White Leghorn, Brown Leghorn, Sussex, New Hampshire, Rhode Island, Ausstralorp, Minorca, Amrox, California Gray, East Lansing, Italian-Partridge-colored, Marans, Barred Rock, Cou Nu Rouge (CNR), GF30, ISA.

In another preferred embodiment, the avian embryonic stem cells according to the present invention are duck embryonic stem cells. In a more preferred embodiment, the duck embryonic stem cells according to the invention are preferably selected from the group consisting of Pekin or Muscovy strain. The duck embryonic stem cells can also from a mulard embryo. As is well known to one of skill in the art mulard embryo can be obtained by crossing a Cairina moschata male with a Anas platyrhynchos female.

Preferably, the avian embryonic stem cells of step a) according to the invention are isolated from freshly laid fertilized eggs, i.e. at a developmental stage named oviposition. Oviposition corresponds to the following development stages according to Eyal-Giladi's classification:
Muscovy duck: stage VII;
Guinea fowl: stage VII-VII I;
Turkey: stage VII-VIII;
Pekin duck: stage VIII;
Chicken: Stage X;
Japanese Quail: stage XI;
Goose: stage XI.

Preferably, the Pekin duck embryonic stem cells are obtained by dissociating embryos at around stage VIII (oviposition) of Eyal-Giladi's classification. If the laid egg collected at oviposition is not enough developed to collect embryonic stem cells, the laid egg may be further incubated between several hours to one or two days to mature the embryo.

Preferably, the Muscovy duck embryonic stem cells are obtained by dissociating embryos at around stage VII (oviposition) of Eyal-Giladi's classification.

Preferably, the chicken embryonic stem cells are obtained by dissociating embryos at around stage X (oviposition) of Eyal-Giladi's classification.

Alternatively, the avian embryonic stem cells of step a) according to the invention are collected from embryo before oviposition. The main limitations encountered before oviposition is the fact that the egg has to be surgically removed from hens and that the amount of embryonic stem cells per embryo is less important. The person skilled in the art will be able to define the timeframe prior egg laying that allows to collect avian embryonic stem cells.

Alternatively, the avian embryonic stem cells of step a) according to the invention may be collected from avian embryo after oviposition up to hatching. The person skilled in the art will be able to define the timeframe after egg laying that allows to collect avian embryonic stem cells. However, it is preferred that the avian stem cells, in particular the avian embryonic stem cells, are not collected after oviposition, in particular from a formed embryo.

According to an embodiment of the present invention the avian embryonic stem cells of step a) of the invention may be a population of embryonic stem cells enriched in primordial germ cells (PGC). Preferably, the avian embryonic stem cells are purified primordial germ cells. In avian species, primordial germ cells arise from the central region of the blastoderm. Then they move to an anterior extra-embryonic site, the germinal crescent, until collected by the vasculature between 2.5 and 5 days of embryonic development to reach the germinal ridge. They colonize the germinal ridge where they eventually differentiate into oocytes or spermatocytes. Methods for isolation of PGCs from donor avian embryos can easily be performed by the person skilled in the art. According to an embodiment, PGCs are collected from embryonic blood collected from the dorsal aorta of a chicken embryo at stage 12-14 of Hamburger & Hamilton's classification (Hamburger & Hamilton (1951) J. Morphol. 88: 49-92). In another preferred embodiment, PGCs are collected from the germinal crescent by mechanical dissection of chicken embryo or from the gonads. However, others methods for isolating PGCs are known and can alternatively be used by the person skilled in the art.

By “passage” it is meant the transfer of transplantation of cells, with or without dilution, from one culture vessel to another. It is understood that any time cells are transferred from one vessel to another, a certain portion of the cells may be lost and therefore, dilution of cells, whether deliberate or not, may occur. This term is synonymous with the term “subculture”. The passage number is the number of times the cells in the culture, which grow either in suspension or in adherence, have been sub-cultured or passed in a new vessel.

The term “diploid” refers to cells of the invention which have two copies (2n) of each chromosome, usually one from the mother and one from the father.

The cell lines of the invention are “continuous” because they have the characteristics to be cultured in vitro over an extended period of time. Advantageously, the cells of the invention are capable of proliferating for at least 50 generations, at least 75 generations, at least 100 generations, at least 125 generations, at least 150 generations, at least 175 generations, at least 200 generations, at least 250 generations. The 250 generations do not constitute a time limit because the cells obtained are still alive and can still be passaged for additional passages.

The factors allowing the growth of the avian stem cells, in particular the avian embryonic stem cells of step a) of the invention are preferably selected from the group consisting of Insulin Growth factor 1 (IGF-1), Ciliary Neurotrophic factor (CNTF), interleukin 6 (IL-6), interleukin 6 receptor (IL-6R), Stem cell Factor (SCF) and Fibroblast Growth Factor (FGF).

The complete culture medium of step a) according to the invention is preferably a “basal culture medium”. The basal culture medium according to the invention is preferably a culture medium with a classical media formulation allowing, by itself, at least cells survival, and even better, cell growth. Examples of basal media according to the invention are BME (basal Eagle Medium), MEM (minimum Eagle Medium), medium 199, DMEM (Dulbecco's modified Eagle Medium), GMEM (Glasgow modified Eagle medium), DMEM-HamF12, Ham-F12 and Ham-F10, Iscove's Modified Dulbecco's medium, MacCoy's 5A medium, RPMI 1640, GTM3, EX-CELL EBx GRO-I serum-free medium (SAFCBiosciences), or HyClone CDM4Avian medium. Preferably, basal medium according to the invention comprises inorganic salts such as CaCl2, KCl, NaCl, NaHCO3, NaH2PO4, MgSO4, etc., amino-acids, vitamins such as thiamine, riboflavin, folic acid, D-Ca panthothenate, etc. and others components such as glucose, beta-mercaptoethanol, sodium pyruvate. Preferably, the basal medium according to the invention is a synthetic medium. In addition, the basal medium of the invention may be complemented with additives selected from the group consisting of 0.1 to 5 mM of L-glutamine, preferably between 2 to 3 mM of L-Glutamine; 0.05 to 2 mM of sodium pyruvate, preferably between 0.1 mM to 1 mM of sodium pyruvate; 0.1 to 2.5% of non-essential amino-acids, preferably around 1% of non-essential amino-acids; 0.1 to 2.5% of vitamins, preferably around 1% of vitamins, 0.05 to 5 mM of beta-mercaptoethanol, preferably around 0.16 mM of beta-mercaptoethanol; and protein hydrolysate of non-animal origin.

During the process of establishment of avian stem cells, in particular avian embryonic stem cell according to the invention, the cells are preferably cultured on a layer of feeder cells. Preferably, feeder cells are animal cells or cell lines cultured for the purpose of culturing avian stem cells, in particular avian embryonic stem cells. Alternatively, the feeder cells can be substituted with extra-cellular matrix and bound growth factors. Feeder matrix will thereafter refer to either feeder cells or extra-cellular matrix. A feeder matrix as used herein is preferably constructed in accordance with procedures known in the art. It is preferred that the feeder matrix be preconditioned. The term “preconditioned” refers to the feeder matrix which is cultured in the presence of media for a period of time prior to the depositing of avian stem cells, in particular avian embryonic stem cells, e.g. a time sufficient to initiate and establish production of, for example, growth factors or other factors by the feeder matrix; usually a feeder matrix is preconditioned by culturing the feeder matrix by itself for one to two days prior to the depositing of avian stem cells, in particular avian embryonic stem cells, in contact with the feeder matrix. The feeder cells preferably comprise mouse fibroblast cells. STO fibroblasts are preferred, but primary fibroblasts are also suitable. It is contemplated that feeder matrices comprising cells from other murine species (e.g. rat); other mammalian species (e.g. ungulate, bovine, porcine species); or avian species (e.g. Gallinacea, chicken, turkey, duck, goose, quail, pheasant) may also be used. In another embodiment, feeder cells of the invention may be transfected with expression vector(s) allowing for example the constitutive expression of growth factors such as avian SCF in STO cells. Thus, this feeder may produce the factor in a form which is soluble and/or attached in the plasma membrane of the cells. Thus, the culturing process of the present invention may optionally comprise establishing a monolayer of feeder cells. Feeder cells are mitotically inactivated using standard techniques. For example, the feeder cells may be exposed to X or gamma radiation (e.g. 4000 Rads of gamma radiation) or may be treated with Mitomycin C (e.g. 10 μg/ml for 2-3 hours). Mono-layers may optionally be cultured to about 80% confluency, preferably to about 90% confluency, and more preferably about 100% confluency. While configuration of the feeder cells as a monolayer is the preferred configuration for the culture, any suitable configuration is contemplated to be within the scope of the present invention. Thus, for example, layers, mono-layers, clusters, aggregates or other associations or groupings of feeder cells are contemplated to fall within the scope of the present invention and are particularly contemplated to fall with the meaning of the term “matrix”.

In an embodiment, the culture medium of step a) according to the invention is supplemented with animal serum. The animal serum preferably used is fetal animal serum. Fetal bovine serum is preferred. Serum from other animal species (e.g. chicken, horse, porcine, ungulate, etc.) may also be used. The final concentration of animal serum in the culture medium is preferably comprised between approximately 1 to 25%, preferably between 5% to 20%, more preferably between 8% and 12%.

In another embodiment, the culture medium of step a) according to the invention is not supplemented or does not comprise animal serum.

The modification of the culture medium in step b) of the process for obtaining avian stem cells, in particular avian embryonic stem cells, according to the invention, so as to obtain progressive or total withdrawal of growth factors, serum and feeder layer, can be made simultaneously, successively or separately. The sequence of the weaning of the culture medium may for example be chosen among:
feeder layer/serum/growth factors;
feeder layer/growth factors/serum;
serum/growth factors/feeder layer;
serum/feeder layer/growth factors;
growth factors/serum/feeder layer;
growth factors/feeder layer/serum.

In a preferred embodiment, the sequence of the weaning is growth factors/feeder layer/serum. In a preferred embodiment, the withdrawal of additives such as sodium pyruvate, non-essential amino acids (NNEA), vitamins, yeastolate are performed after the weaning of feeder layer and before the weaning of serum. Preferably, the withdrawal of yeastolate is performed after the withdrawal of sodium pyruvate, NNEA and vitamins.

### Culture of Avian Stem Cells

The established avian stem cells, in particular avian embryonic stem cells, according to the invention by the method as disclosed above have the characteristics to grow in a serum free medium which means preferably free of exogenous growth factors and animal serum and without feeder cells.

The avian stem cells, in particular avian embryonic stem cells, according to invention, preferably duck and chicken embryonic stem cells, can be in vitro cultured over a considerable period of time. Advantageously, suspension of avian stem cells, in particular avian embryonic stem cells, obtained by the method according to the invention disclosed above, are capable to proliferate for at least 50 generation, at least 75 generation, at least 100 generation, at least 125 generation, at least 150 generation, at least 175 generation, at least 200 generation, at least 250 generation.

Preferably, the avian stem cells, notably the avian embryonic stem cells, of the invention may proliferate indefinitely in a serum free medium.

The expression “serum-free medium” (SFM) according to the invention means a cell culture medium ready to use, which preferably does not require animal serum addition allowing cells survival and cell growth. This medium is not necessary chemically defined, and may contained hydrolysates of various origin such as plant or yeast. Preferably, said SFM are “non animal origin” qualified, which means that it does not contain components of animal or human origin. In SFM, the native serum proteins are preferably replaced by recombinant proteins. Alternatively, SFM medium according to the invention preferably does not contain protein and/or are chemically defined medium. SFM medium preferably present several advantages: (i) the regulatory compliance of such media; (ii) the optimization of the purification process; (iii) the better reproducibility in the process due to the better defined medium. Examples of commercially available SFM media are: VP SFM (InVitrogen Ref 11681-020, catalogue 2003), Opti Pro (InVitrogen Ref 12309-019, catalogue 2003), Episerf (InVitrogen Ref 10732-022, catalogue 2003), Pro 293 S-CDM (Cambrex ref 12765Q, catalogue 2003), LC17 (Cambrex Ref BESP302Q), Pro CHO 5-CDM (Cambrex ref12-766Q, catalogue 2003), HyQ SFM4CHO (Hyclone Ref SH30515-02), HyQ SFM4CHO-Utility (Hyclone Ref SH30516.02), HyQ PF293 (Hyclone ref SH30356.02), HyQ PF Vero (Hyclone Ref SH30352.02), Excell 293 medium (SAFC Biosciences ref 14570-1000M), Excell 325 PF CHO Protein free medium (SAFC Biosciences ref 14335-1000M), Excell VPRO medium (SAFC Biosciences ref 14560-1000M), Excell 302 serum free medium (SAFC Biosciences ref 14312-1000M), Excell 65319, Excell 65421, Excell 65625, Excell 65626, Excell 65627, Excell 65628, Excell 65629 (JRH Biosciences), Excell MDCK SFM (SAFC-Biosciences Ref. 14581C), Excell MDCK Prod (Ref. M3678), Gene Therapy Medium 3 (animal component free) (SIGMA-Aldrich, ref. G-9916 or Excell GTM-3) (hereinafter named G9916 medium), HYQ CDM4 HEK-293 (Hyclone Ref. SH30859), HYQ SFM4 HEK-293 (HYCLONE Ref. SH30521), AEM (InVitrogen), EX-CELL EBx GRO-I serum-free medium (SAFC Biosciences), CDM4Avian medium (Hyclone).

The serum-free medium may be supplemented with at least one ingredient selected from the group consisting of amino-acids, lipids, fatty acids, cholesterol, vitamins, carbohydrates, protein hydrolyzates of non-animal origin, and a mixture thereof.

By way of example, the amino-acids added to the medium may be selected from the group consisting of asparagine and glutamine, or a mixture thereof.

By way of example, the carbohydrates added to the medium may be selected from the group consisting of D-glucose, D-sucrose and D-galactose or a mixture thereof. According to a more preferred embodiment, the carbohydrate added is D-glucose.

By way of example, the lipids are selected from the group consisting of cholesterol, steroids, and fatty acids such as palmitic acid, palmitoleic acid, stearic acid, oleic acid, linoleic acid, linolenic acid, and their derivatives, or a mixture thereof.

The medium may contain auxiliary substances, such as buffer substances like sodium bicarbonate, oxidation stabilizers, stabilizers to counteract mechanical stress, or protease inhibitors.

The cultivation vessel according to the invention is preferably selected from the group consisting of continuous stirred tank bioreactor, Wave™ Bioreactor, Bello™ bioreactor, spinner flask, flask and a cell factory. According to a preferred embodiment, the cultivation vessel is a continuous stirred tank bioreactor allowing the control of temperature, aeration, pH and other controlled conditions and which is equipped with appropriate inlets for introducing the cells, sterile oxygen, various media for cultivation and outlets for removing cells and media and means for agitating the culture medium in the bioreactor.

### Food Product

Preferably, the food component according to the invention is selected from the group consisting of anti-foaming agents, emulsifiers, firming agents, gelling agents, humectants, mineral salts, stabilizers, thickeners, and texturizing agent.

Preferably, the anti-foaming agent is selected from the group consisting of polyethylene glycol and triethyl citrate.

Preferably, the emulsifier is selected from lecithin, sorbian monostearate and ammonium slats of phosphatidic acids.

The firming agent is preferably selected from the group consisting of calcium chloride, calcium gluconate and calcium sulfate.

The gelling agent is preferably selected from the group consisting of agar, calcium alginate and carrageenan.

The humectant is preferably selected from the group consisting of glycerin, glycerol, lactitol and oxidized polyethylene; the mineral salt is cupric sulfate.

Preferably, the stabilizer is selected from the group consisting of xanthan gum, guar gum and bleached starched.

The thickener is preferably selected from the group consisting of tannins, sodium alginate and pectin.

Preferably, the texturizing agent is selected from the group consisting of phosphates, sodium tripolyphosphate, sodium hexametaphosphate and sodium pyrophosphate.

In an embodiment of the present invention, the food product according to the invention further comprises at least one supplementary food component. Preferably, the supplementary food component is selected from the group consisting of vitamins, minerals, fibers, vegetable oil, fatty acids, amino acids, flavoring agents, colorants, antioxidants, sweeteners, flavor enhancers, acidifiers, preservatives, sequestrants, seasonings, sugar, flour, prebiotics, salt, water, and antimicrobials.

Preferably, the vitamin is selected from the group consisting of B vitamins including niacin, vitamin C and vitamin E.

The flavoring agent is preferably selected from the group consisting of oleoresin and aquaresins.

The colorant is preferably selected from the group consisting of curcumin, brilliant blue, tartrazine and ferrous gluconate.

Preferably, the antioxidant is selected from the group consisting of nitrates, nitrites, butylated hydroxyanisole, ascorbyl palmitate and calcium ascorbate.

Preferably, the sweetener is selected from the group consisting of sorbitol, alitame, aspartame, saccharin, calcium saccharin and corn syrup.

The flavor enhancer is preferably selected from the group consisting of acetic acid, citric acid and fumaric acid.

The acidifier is preferably lactic acid.

The preservative is preferably selected from the group consisting of sodium nitrate, benzoic acid, sodium benzoate, tocopherols, ascorbic acid, niacin, riboflavin and thiamine.

Preferably, the sequestrant is potassium gluconate.

The seasoning is preferably selected from the group consisting of spices or oleoresins extracted from them, herbs, vegetables, essential oils, sodium nitrate, water, salt, sugar, and flavors.

Preferably, the antimicrobial is selected from the group consisting of lactic acid, citric acid, acetic acid, sodium diacetate, acidified sodium chloride or calcium sulfate, cetylpyridinium chloride, activated lactoferrin, sodium or potassium lactate, or bacteriocins such as nisin.

Preferably, the food product according to the invention does not comprise animal component in addition to the avian stem cells.

It should be clear that the food product according to the invention is neither a bird/bird part nor an egg, in particular not a fertilized egg, such as a freshly fertilized egg or a balut. More generally, it should be clear that the food product according to the invention is not a naturally occurring product, it is a manufactured product.

Besides, the avian stem cells, in particular the avian embryonic stem cells, comprised in the food product according to the invention preferably consist of at least 60%, at least 70%, at least 80%, at least 90, at least 95% of undifferentiated cells. Preferably, the food product according to the invention comprises less than less than 50%, 40%, 30%, 20%, 10%, 5%, 2%, or 1% of differentiated cells of avian origin (calculated as the number of differentiated cells of avian origin/total number of cells of avian origin (differentiated+undifferentiated)).

Preferably, the food product according to the invention is suitable for human and/or non-human animal consumption.

Preferably, the food product according to the invention has organoleptic properties close to meat product's organoleptic properties. Method to determine organoleptic properties are well known in the art such as for example use of a panelist or artificial means. More preferably, the food product according to the invention has the organoleptic properties of a meat product selected from the group consisting of beef, buffalo, bison, calf, goat, ham, horse, kangaroo, lamb, moose, mutton, pork, bacon, rabbit, venison, chicken, duck, emu, goose, guinea fowl, ostrich, partridge, pheasant, pigeon, quail, and turkey.

Preferably, the food product according to the invention comprises from 5 to 30% (dry w/w) proteins.

Preferably, the food product according to the invention comprises from 1 to 5% (dry w/w) lipids.

Preferably, the food product according to the invention comprises at least 1% (dry w/w) minerals, including iron.

Preferably, the food product according to the invention comprises at least 1 essential amino acids selected from phenylalanine, leucine, methionine, lysine, isoleucine, valine, threonine, tryptophan, and histidine. More preferably the food product according to the invention comprise at least 2, 3, 4, 5, 7, and 8 essential amino acids.

The food product according to the invention is preferably in a unit form of at least grams, at least 10 grams, at least 15 grams, at least 25 grams, at least 50 grams, at least 70 grams, at least 80 grams, at least 90 grams, at least 100 grams.

Preferably also, the food product according to the invention is in a form selected from the group consisting of a fresh product, a dried product, a frozen product, a product which has been cooked, pickled or smoked or in a form incorporated in a processed food product, in particular a soup, a stew, a sausage, a spread, a puree, a biscuit, dried granules, tablets, capsules, powder, pasta box, pizzas, ready-to-eat meals, sandwiches or nuggets.

The food product according to the invention can be used as a food supplement, in particular a protein supplement, preferably for the elderly or as a sport or workout supplement. Preferred forms for the food supplements, are the liquid form, in particular shakes, or the powder form.

The food product according to the invention is preferably packed in a commercial packaging suitable for distribution to an end consumer. The packaging according to the invention may be any suitable packaging known to the person skilled in the art such as bag, boxes, cans, cartons, coated paper, flexible packaging, pallets, wrappers, trays, bottles, glass containers, cup, envelope, pizza box, microwave box, pasta box, jar.

### Preparation of a Food Product

Preferably, the food product according to the invention is prepared by a method comprising a step of mixing avian stem cells, in particular avian embryonic stem cells, according to the invention with at least one food component according to the invention.

In one embodiment, the method for the preparation of the food product according to the invention further comprises at least one step of culturing the avian stem cells, in particular the avian embryonic stem cells, according to the invention in a culture medium in vitro. Preferably, the method of preparing the food product according to the invention comprises the following steps:
at least one step of culturing avian stem cells, in particular avian embryonic stem cells, in a culture medium in vitro;
collecting the avian stem cells;
mixing the avian stem cells with at least one food component according to the invention to obtain a food product.

Preferably, the step of culturing avian stem cells, in particular avian embryonic stem cells, does not substantially lead to the differentiation of the cells, i.e. leads to less than 50%, 40%, 30%, 20%, 10%, 5%, 2%, or 1% of differentiated cells (calculated as the number of differentiated cells/total number of cells (differentiated+undifferentiated)).

The step of culturing avian stem cells, in particular avian embryonic stem cells, in a culture medium in vitro according to the invention can occur before the incorporation of a food component according to the invention or after the incorporation of a food component according to the invention.

In an embodiment of the invention, the method further comprises the addition of at least one additional food component according to the invention.

Preferably, the method further comprises at least one step of food processing in order to provide the food product in a consumption form. The food processing step according to the invention may be done by any means well known to the person skilled in the art to transform food product. By way of example, the food processing step can be selected from the group consisting of solidification, pressing, drying, freeze-drying, freezing, boiling, cooking, smoking, irradiating, homogenizing, under pressure cooking, canning, pasteurization, and packaging.

Preferably, the food product is processed in a form selected from the group consisting of fresh product, a dried product, a frozen product, a product which has been cooked, pickled or smoked or in a form incorporated in a processed food product, in particular a soup, a stew, a sausage, a spread, a puree, a biscuit, dried granules, tablets, capsules, powder, pasta box, pizzas, ready-to-eat meals, sandwiches or nuggets.

The present invention will be further explained with the following non-limiting Examples.

### EXAMPLES

### Example 1

### 1. Methods

Duck embryonic stem cells are stored in cryo-vials at −80° C. The cells are thawed and used to seed 3 L-Erlenmeyers in a serum-free liquid HyClone CDM4Avian medium (GE Healthcare) supplemented with 2.5 mM I-glutamine. The cells are cultured using an orbital shaker (150 rpm) at 37° C., under 7.5% CO₂ atmosphere. The cells are then collected as pellets by centrifugation (3×100 g) in 1 L-bottles.

250 g of the collected cells are then mixed with ½ egg and ½ table spoon of wheat flour, processed as balls and cooked in a pre-heated oven at 180° C. until the inner temperature of the balls reaches 70° C., to obtain a food product. Similar meatballs are prepared with minced duck.

### 2. Results

The nutritional qualities of the product are evaluated and compared with the nutritional profile of the duck meatballs.

The cell-based meatballs of the invention have a similar nutritional profile, in particular as regards the protein content, as the duck meatballs

### Example 2

The cells are prepared as indicated in Example 1.

The following food products (nuggets) are prepared:

| | Vegetarian control | Example |
|---|---|---|
| Ingredient | (g) | (g) |
| Hydrated textured soybean protein | 75.0 | 60.0 |
| Avian stem cells | — | 15.0 |
| Nugget crumb preparation | 10.0 | 10.0 |
| Vegetable oil | 5.0 | 5.0 |
| Water | 10.0 | 10.0 |
| Total | 100.0 | 100.0 |

After being fried, the food product according to the invention presents with a protein content similar to that of the control but offers organoleptic properties closer to a traditional chicken nugget than the control does.

1. A method for the preparation of a food product, comprising:
at least one step of culturing avian stem cells in a culture medium in vitro;
collecting the avian stem cells; and
mixing the avian stem cells with at least one food component to obtain a food product.

2. The method according to claim 1, wherein the avian stem cells are isolated from embryo.

3. The method according to claim 1, wherein the avian stem cells are from duck and/or chicken.

4. The method according to claim 1, wherein the culture medium is a serum-free medium.

5. The method according to claim 1, wherein the culture medium does not comprise an animal component.

6. The method according to claim 1, wherein the step of culturing avian stem cells does not substantially lead to the differentiation of the cells.

7. The method according to claim 1, further comprising the addition of at least one additional food component.

8. The method according to claim 1, further comprising at least one step of food processing selected from the group consisting of solidification, pressing, drying, freeze-drying, freezing, boiling, cooking, smoking, irradiating, homogenizing, under pressure cooking, canning, pasteurization, and packaging.

9. The method according to claim 1, wherein the food component is selected from the group consisting of anti-foaming agents, emulsifiers, firming agents, gelling agents, humectants, mineral salts, stabilizers, thickeners, and texturizing agent.

10. The method according to claim 1, wherein the food product has organoleptic properties close to a meat product's organoleptic properties, wherein the meat product is selected from the group consisting of beef, buffalo, bison, calf, goat, ham, horse, kangaroo, lamb, moose, mutton, pork, bacon, rabbit, venison, chicken, duck, emu, goose, guinea fowl, ostrich, partridge, pheasant, pigeon, quail, and turkey.

11. A method for the preparation of a food product, comprising a step of mixing avian stem cells with at least one food component.

12. The method of claim 11, further comprising at least one previous step of culturing the avian stem cells in a culture medium in vitro.

13. The method of claim 11, wherein the avian stem cells are isolated from an embryo.

14. The method of claim 11, wherein the avian stem cells are from duck and/or chicken.

15. The method according to claim 11, wherein the food product has organoleptic properties close to a meat product's organoleptic properties, wherein the meat product is selected from the group consisting of beef, buffalo, bison, calf, goat, ham, horse, kangaroo, lamb, moose, mutton, pork, bacon, rabbit, venison, chicken, duck, emu, goose, guinea fowl, ostrich, partridge, pheasant, pigeon, quail, and turkey.

16. The method according to claim 11, wherein the food product is obtained in a form selected from the group consisting of a fresh product, a dried product, a frozen product, a product which has been cooked, pickled or smoked or in a form incorporated in a processed food product.

17. The method according to claim 1, wherein the culture medium does not comprise animal serum.
